(19) United States
(12) Patent Application Publication
(10) Pub. No.: US 2009/0234379 A1
(43) Pub. Date: Sep. 17, 2009

(54) Apparatuses for the performance of a minimally invasive ventral hernia repair

(76) Inventor: Robert D. Rehnke, St. Petersburg, FL (US)

Correspondence Address: Charles Raymond Sidwell, 2032 Dolphin Blvd., South, St. Petersburg, FL, 33707, US

(21) Appl. No.: 12/075,629

(22) Filed: Mar. 14, 2008

(51) Int. Cl. A61B 17/32; A61B 17/94; A61B 17/068

(52) U.S. Cl. 606/190; 600/104; 227/175.1

(57) ABSTRACT

An Apparatus for the performance of a minimally invasive ventral hernia repair. The apparatus including a mechanical blunt dissector, fascial graspers, and a midline fascial stapling device. The mechanical blunt dissector is capable of performing a blunt dissection of the space between the External Oblique (EO) muscle and the Internal Oblique (IO) muscle. The device has a blunt spoonbill shaped set of plates with an open center (for improved endoscopic visualization during the dissection), connected to long arms which connect to a pair of hand grips. However, the blunt dissector may include a tapered blunt distal tip to the spoonbill element—more like a duck's bill—for advancing the mechanical dissector forward into the loose areolar connective tissue that exists between the EO and IO muscles. The apparatus further includes graspers formed of long endoscopic graspers that are modified to grasp and lock on to tissue as thick and tough as rectus fascia. A further inclusion in the present invention is a fascial stapler designed for approximation of the mid line rectus fascia.

### CROSS REFERENCE TO RELATED APPLICATIONS

This application claims the benefit of provisional application No. 60/906,310, filed Mar. 12, 2007.

### BACKGROUND OF THE INVENTION

1. Field of the Invention

The present invention pertains generally to surgical devices for reconstructing an intact abdominal wall, after the development of an incisional ventral hernia, in a minimally invasive manner known as MINIMALLY INVASIVE VENTRAL HERNIA REPAIR (MIVHR).

2. Description of the Prior Art

The most common general surgical procedure performed annually is the laparotomy. Access to the peritoneal cavity through a midline incision and division of the linea alba provides access to the abdominal cavity and its contents. For operations on the stomach, duodenum, and other upper abdominal organs an epigastric incision from xiphoid to umbilicus is used; incisions from umbilicus to pubic symphasis are used for operations performed on pelvic organs such as the sigmoid colon, bladder and uterus. Operations requiring greater exposure, such as hepatic or pancreatic procedures, use midline incisions encompassing the entire length from xiphoid to pubis. Midline abdominal incisions have become the gold standard for access to the abdominal cavity in spite of one significant drawback—post operative incisional hernias. Most studies cite a 10 to 15 percent incidence of hernia formation following laparotomy. This alarming statistic begs the question, why?

What factors lead to ventral, incisional hernias? Certainly surgeons worry about poor technique in closing the abdominal wall, as much has been written on this subject. It seems that a running closure with a large gauge, non absorbable suture that avoids uneven and excessive tension (that could strangulate tissue), is indicated. Complications of healing must be avoided if low hernia rates are expected. Such problems as hematoma, seroma and infection are direct contributors to poor healing of the midline. Post operative ileus or adhesions leading to periodic partial small bowel obstructions cause increased intra abdominal pressure that tests the integrity of the midline repair. Other, indirect causes include: sepsis, hypo proteinemia, and post op pulmonary failure. Certain chronic conditions, such as diabetes, smoking, obesity, and COPD, are felt to lead to increased risk of herniation.

Unbalanced tension in the abdominal wall after closure of the midline is a factor not quite as obvious as those previously mentioned. The muscular anatomy of the abdominal wall creates force vectors that pull the midline apart during movement. The only force holding the insertion of the obliques and the rectus abdominus together is the linea alba, or fascial midline of the anterior abdomen. Once this has been divided by laparotomy incision the forces that balance the muscular tensions of the abdomen will never be the same. There will always be an inherent weakness to the abdominal wall at the midline. Closure at the end of the procedure approximates the left and right sides of the rectus muscle and brings the fascial edges of the linea alba together. However, any imperfection of healing of the seam can lead to a pressure leak that can progress to a fascial defect. Over time the unbalanced pressures of the abdominal wall force vectors act to erode the closure of the abdomen at its weakest spot—the repaired linea alba. In this fashion hernia defects grow over time, explaining why the incidence of hernia diagnosis is greater at ten years post op than that seen in the first two years.

The final factor in hernia formation is poor tissue quality. Patients who develop hernias after laparotomy tend to develop recurrent hernias after hernia repairs; this in large part can be attributed to poor quality tissue. The variation in tensile strength of fascia and the relative thickness of fascia present for suturing is observed clinically. Even when strong sutures are placed correctly, weak tissue allows migration of the suture through the soft tissue which leads to loosening of the closure, separation of the midline, and hernia formation. All other factors being the same, patients with strong tissue will hold the placement of the suture until healing of the midline has occurred and avoid hernia formation.

Over ten percent of all laparotomies will develop ventral hernias leading to over 350,000 ventral hernia repairs per year in the U.S. alone. Despite advances in the art of hernia repair, over the last century, there still is a need for improvement. Historically ventral hernias were treated with primary closure, a practice that achieved no greater than a 50% success rate. Large hernias were closed with a variety of cadaveric or autologous tissue grafts or flaps without much better success. Finally techniques using synthetic mesh, as an inlay or on lay, lead to improved outcomes. Unfortunately covering fascial defects with mesh often led to complications such as infection, entero-cutaneous fistulas, chronic pain, recurrent partial small bowel obstructions, and still unacceptably high recurrence rates approaching 25%.

French surgeons Rives' and Stopa developed an approach in the early 1970's, which addressed these concerns and is still widely used in Europe today. The “French” procedure as it came to be known consists of placement of a non-absorbable mesh over closure of the posterior rectus sheath. Once the hernia defect is debrided of scar tissue, the posterior rectus sheath is separated from the anterior rectus sheath and muscle, and a space is dissected behind the rectus muscles. Once the posterior rectus sheath is pulled together and sutured, a mesh is cut to size and pulled into place by stay sutures which are passed behind the muscle and anchored to the three layers of the abdominal wall—lateral to Spigal's line. The rectus muscles and anterior rectus sheath is then closed at the midline over the mesh layer. The key to this procedure is a re-enforcing layer of mesh within a mid line closure of the defect. It avoids exposure of the bowel to mesh and restores the abdominal muscles to their natural form and function. It is limited in its effectiveness by the fact that it closes the midline under tension (relying on the strength of the mesh layer to overcome this weakness). This breaks a cardinal rule of hernia repair and requires bridge gap placement of absorbable mesh when the posterior and anterior rectus fascial layers can not be safely brought together. This, in effect, creates a controlled diastasis and limits the success of the repair. As a result, the French repair has had limited popularity in the U.S.

Modern advances in the treatment of ventral hernia were realized towards the end of the 20^{th} century when two novel techniques were developed: Laparoscopic Ventral Hernia Repair (LVHR) and Components Separation Technique. The former evolved from the expansion of the minimally invasive laparoscopic movement in General Surgery. It was aided by a new advancement in prosthetic materials—dual plane mesh. It is performed through a “closed” laparoscopic technique utilizing the placement of an overlapping mesh inlay patch of the fascial defect—a so called “no tension” repair. The mesh, which has a smooth inner layer to prevent adhesions to bowel and a textured outer layer to anchor to the abdominal wall, is designed to overlap the edge of the defect by three to five centimeters. The Components separation Technique (CST) was developed by the noted plastic surgeon Oscar Ramirez in 1990. It utilizes a midline approach with extensive undermining to raise skin flaps that expose the anterior abdominal wall. A long relaxing incision just lateral to Spegil's line separates the external oblique (EO) from the inner three layers of the abdominal wall (internal oblique (IO), transversus abdominus, and transversalis fascia). Blunt dissection in the fascial cleft between EO and IO layers allows the mobilization of composite myofascial flaps towards the midline for primary closure without undue tension. This procedure closes the defect with dynamic living tissue that restores abdominal function and aesthetics. Both techniques in experienced hands have been demonstrated in the surgical literature to be safe and effective (recurrence rates approaching 5%).

Both Laparoscopic and CST, repairs have their advantages and draw backs. LVHR is noninvasive and shown to have shortened hospital stays. It has decreased morbidity and mortality when compared to open ventral hernia techniques. However, it requires placement of expensive and potentially dangerous prosthetic mesh inside the peritoneal cavity. Additionally, it does not truly repair the abdominal wall; but instead patches the defect. Without closure of the rectus muscles to their midline approximation there can not be restoration of abdominal function and aesthetics. CST restores form and function without the need for intra-peritoneal mesh, but is an invasive procedure, which is time consuming and difficult to perform, with high rates of wound complications. This explains why the clear majority of hernia repairs are still performed with some form of prosthetic mesh repair.

Advances in hernia repair since the turn of the century have focused on improving the technology of mesh design. The development of modern mesh materials has introduced the concept of lightweight mesh and bi laminar mesh designs. These developments address some of the problems experienced with mesh over the last fifty years. In addition to recurrence of the hernia, mesh repairs can be complicated by stiffening and contracture of the prosthetic device which leads to discomfort and reduced flexibility of the abdominal wall. Additionally, intra peritoneal positioning of prosthetic can lead to intestinal adhesion, fistualization and infection. These modern prosthetics have a lightweight, flexible abdominal wall layer and a resorbable biomaterial inner layer to prevent adhesions. Two examples of modern mesh prosthetics are Ethicon's Proceed, which is a lightweight polypropylene mesh with an oxidized regenerated cellulose inner layer, and Sofradim's Parietex Composite mesh, which is woven polyester with an inner collagen based layer to prevent adhesions. This new generation of prosthetics holds the promise of safe intra peritoneal mesh placement.

A new approach to ventral hernia repair has been proposed for the 21^{st} century in the present invention. The present invention takes advantage of the best principles for ventral hernia repair from the leading techniques of the last quarter century. The present invention starts with the understanding that the midline should be re approximated and reinforced with a retro fascial mesh as taught by Rives. The present invention however precedes this step by fascial components release, as taught by Ramirez, to allow closure of the midline without tension. This however is performed with the technology of endoscopic surgery, avoiding open dissection of skin flaps to expose the abdominal wall for open fascial partition. This minimally invasive approach to components separation technique (CST) not only avoids difficult and time consuming open dissection, but preserves peri umbilical vascular perforators and therefore avoids the high rate of wound complications associated with the open technique. Laparoscopic techniques are used to close the midline and reinforce the repair with a modern mesh that simulates an intact rectus sheath. This culmination in ventral hernia repair advances is referred to as MINIMALLY INVASIVE VENTRAL HERNIA REPAIR (MIVHR).

Accordingly, there is a need for improved surgical instruments for use in surgery for minimally invasive surgical ventral hernia repair to overcome the aforementioned disadvantages in the prior art.

The use of surgical methods and instruments of known designs and configurations is known in the prior art. More specifically, known designs and configurations heretofore devised and utilized are known to consist basically of familiar, expected, and obvious structural configurations and methods, notwithstanding the myriad of designs encompassed by the crowded prior art which has been developed for the fulfillment of countless objectives and requirements.

While these devices fulfill their respective, particular objectives and requirements, the aforementioned patents do not describe a method of Minimally Invasive Ventral Hernia Repair (MIVHR) that is an improvement on its predecessor the laparoscopic patch inlay through Components Separation Technique (CST) using minimally invasive techniques and a surgical apparatus for approximation of the midline and reconstruction of an intact Prosthetic Rectus Sheath.

Therefore, it can be appreciated that there exists a continuing need for a new and improved surgical method and apparatus for use in ventral hernia repair in a minimally invasive manner. In this regard, the present invention substantially fulfills this need.

### DESCRIPTION OF THE INVENTION

In view of the foregoing disadvantages inherent in the known surgical methods for closure of abdominal incisional ventral hernias, the present invention provides for instrumentation which enables a minimally invasive ventral hernia repair. The novel instruments required for such a procedure are as follows: a mechanical blunt dissector, fascial graspers, and a midline fascial stapling device.

The mechanical blunt dissector is provided for performing a blunt dissection of the space between the External Oblique (EO) muscle and the Internal Oblique (IO) muscle. The device has a blunt spoonbill shaped set of plates with an open center (for improved endoscopic visualization during the dissection), connected to long arms which connect to a pair of hand grips. A hinge point of the two long arms joins the hand grips to the spoonbill dissector plates for blunt dissection inside the body.

An alternate embodiment of the blunt dissector includes a tapered blunt distal tip to the spoonbill element—more like a duck's bill. The tip is useful in advancing the mechanical dissector forward into the loose areolar connective tissue that exists between the EO and IO muscles.

A further alternative embodiment of the blunt dissector includes an elongate tubulare structure. The elongate tubular structure separates the hinge of the hand grip arms from the hinge of the spoonbill dissector plate. The tube contains a mechanical coupling device to transmit the separating forces of the hand grip arms to the spreader plates without movement at the incision site.

An additional embodiment of the blunt dissector includes means for mechanically coupling of the spoonbill plates to their spreader arms.

The present invention further includes fascial graspers. The Graspers are long endoscopic graspers that are modified to grasp and lock on to tissue as thick and tough as rectus fascia. The graspers have five sturdy sharp teeth that constitute a pair of stout jaws.

A further inclusion in the present invention is a fascial stapler. The fascial stapler is designed for approximation of the mid line rectus fascia.

The present invention includes several apparatus and method of use as previously described as well as subsequently indicated; a mechanical dissector, a grasper, and a stapler each comprised of a plurality of components.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved surgical apparatus and method of use which has all the advantages of the prior art a surgical novel instruments required for such a minimally invasive ventral hernia repair including a mechanical blunt dissector, fascial graspers, and a midline fascial stapling device and none of the disadvantages.

It is another object of the present invention to provide a new and improved surgical apparatus and method of use which has all the advantages of the prior art a surgical novel instruments required for such a minimally invasive ventral hernia repair including a mechanical blunt dissector, fascial graspers, and a midline fascial stapling device which may be easily and efficiently manufactured and marketed.

It is a further object of the present invention to provide a new and improved a a new and improved surgical apparatus and method of use which has all the advantages of the prior art a surgical novel instruments required for such a minimally invasive ventral hernia repair including a mechanical blunt dissector, fascial graspers, and a midline fascial stapling device which is of a durable and reliable construction.

An even further object of the present invention is to provide a a new and improved surgical apparatus and method of use which has all the advantages of the prior art a surgical novel instruments required for such a minimally invasive ventral hernia repair including a mechanical blunt dissector, fascial graspers, and a midline fascial stapling device which is susceptible of a low cost of manufacture with regard to both materials and labor.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings.

FIG. 1 illustrates a Midline hernia defect.

FIG. 2 illustrates a mechanical dissector comprising the present invention

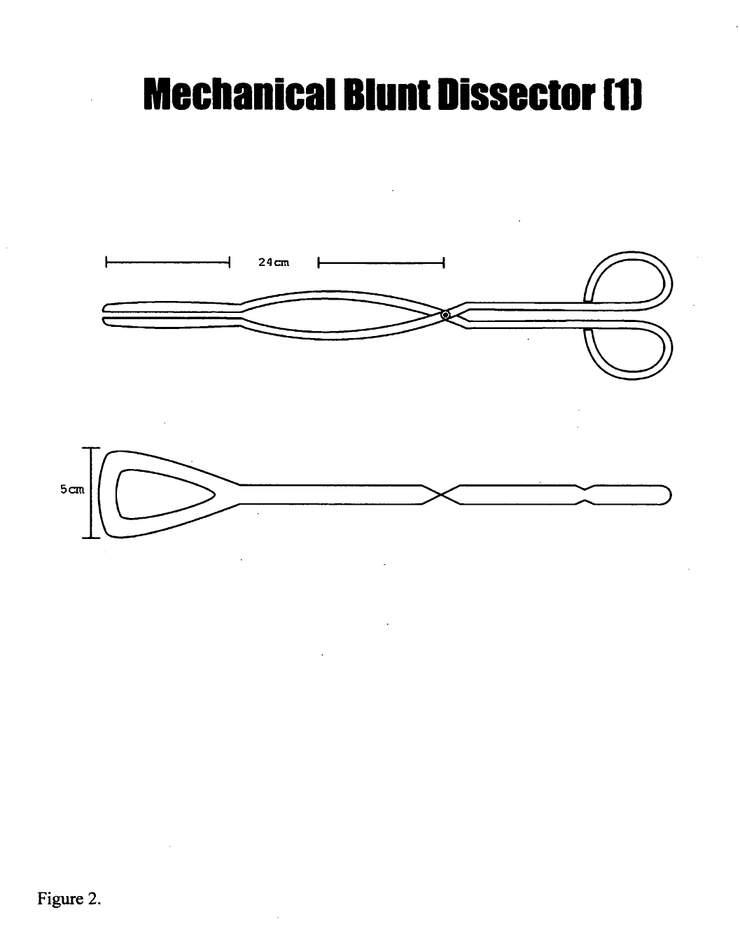

FIG. 3 illustrates an alternative embodimentof the mechanical dissector comprising the present invention.

FIG. 4a. illustrates the blunt dissection external from internal oblique muscle.

FIG. 4b. illustrates an alternative view of FIG. 4a.

FIG. 5 illustrates an endoscopic release of the abdominal components comprising the present invention.

FIG. 6 illustrates the fascial graspers comprising the present invention.

FIG. 7 illustrates laparoscopic approximation of the midline rectus fascia with fascial graspers comprising the present invention.

FIG. 8 illustrates a laparoscopic midline fascial stapler comprising the present invention.

FIG. 9 illustrates a laparoscopic closure of the midline with stapling device comprising the present invention.

FIG. 10 illustrates a reconstructed rectus sheath after endoscopic placement and anchorage.

FIG. 11 illustrates laparoscopic anchorage of prosthetic mesh to four layers of the lateral abdominal wall.

The same reference numerals refer to the same parts throughout the various Figures.

### DESCRIPTION OF THE PREFERED EMBODIMENT

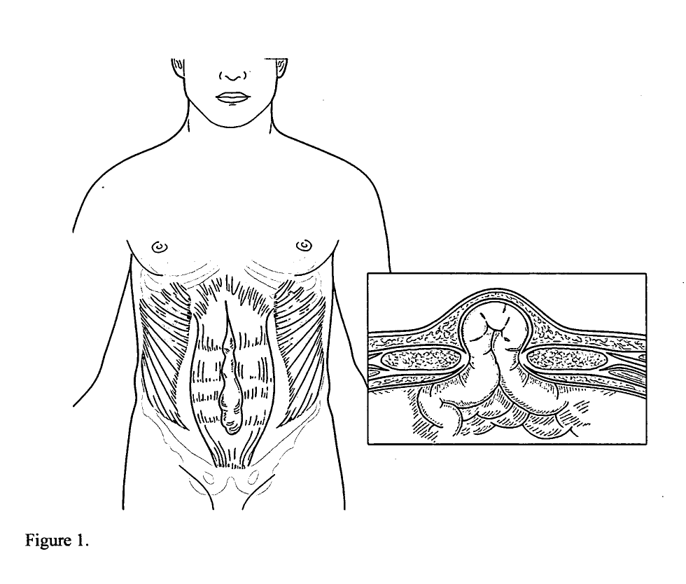

With reference now to the drawings, and in particular to FIG. 1 thereof, the preferred embodiment of the new and improved set of instrument instruments and method of their use in laparoscopic minimally invasive ventral hernia repair embodying the principles and concepts of the present invention and generally designated by the reference numeral 10 will be described.

The present invention comprises a blunt dissector 10. The dissector is a mechanical blunt dissector capable of performing a blunt dissection of a space 12 between the External Oblique (EO) muscle 14 and the Internal Oblique (IO) muscle 16. The dissection is performed through a minimal incision 18 in the inguinal region.

The dissector includes a proximal end 20 and distal end 22 operable associates at a hinge point 24.

The distal end 22 of the dissector includes a blunt spoonbill shaped set of plates 30. the plates have an open center 32. the open center provides for improved endoscopic visualization during the dissection.

The proximal end 20 of the dissector is connected to the distal end 22 by elongate arms 34. The proximal end 20 includes a pair of hand grips. The hinge point 24 is formed in the elongate arms 34 such that in use the handles are kept outside a body allowing the spoonbill dissector plates to be positioned within a body for blunt dissection inside the body.

Between the distal end and hinge point is provide a joining region of the arms to the grips has a narrow waist to allow wide spreading of the arms through a minimal incision. The arms are arched to give strength to the device which can be made of metal or a strong polymer. An alternate design includes a tapered blunt distal tip 40 to the spoonbill element not unlike like a duck bill shape. This shape is useful in advancing the mechanical dissector forward into the loose areolar connective tissue that exists between the EO and IO muscles. Once engaged into this tissue the device is spread open and the two muscle layers are separated bluntly. The instrument can be moved back and forth in a lateral motion, then advanced further into the space between the muscles and opened again to further bluntly dissect open the potential space between the EO and IO muscles. This is repeated over and over again until the limits of the dissection are reached (the semi-lunar line medially, infra mammary fold region superiorly, lumbar region laterally, and the inguinal region inferiorly).

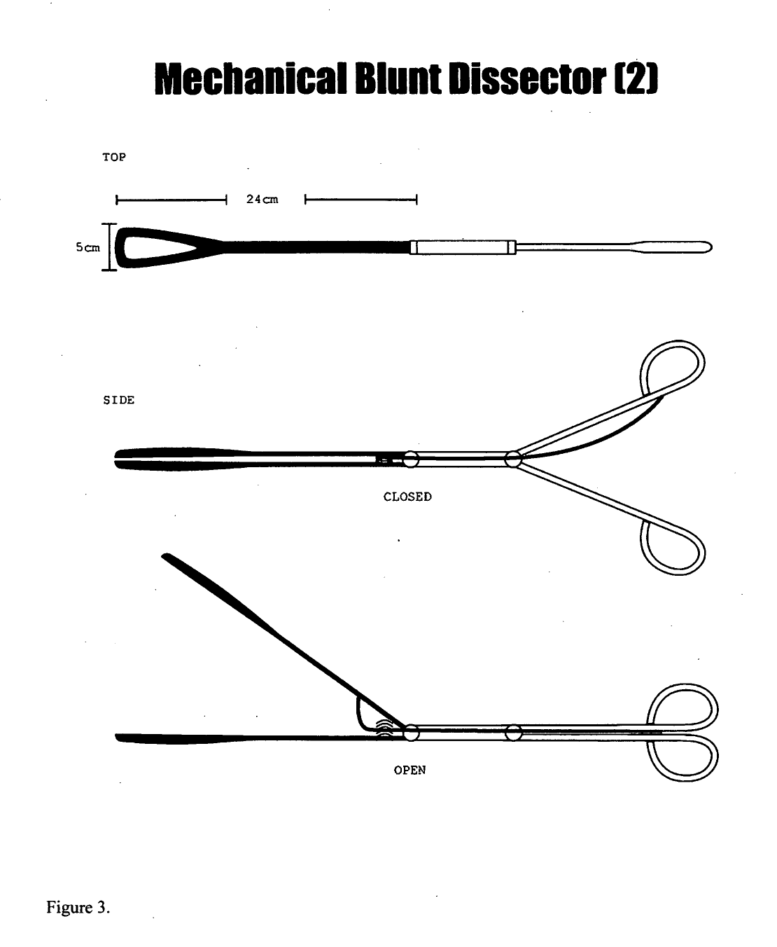

FIG. 3 shows an alternate embodiment of the blunt dissector including separating means for the hinge of the hand grip arms from the hinge of the spoonbill dissector plate arms with an elongated tubular structure. This tube contains a mechanical coupling device to transmit the separating forces of the hand grip arms to the spreader plates without movement at the incision site. This allows for a smaller incision site in the inguinal region and better maneuverability of the instrument through this incision.

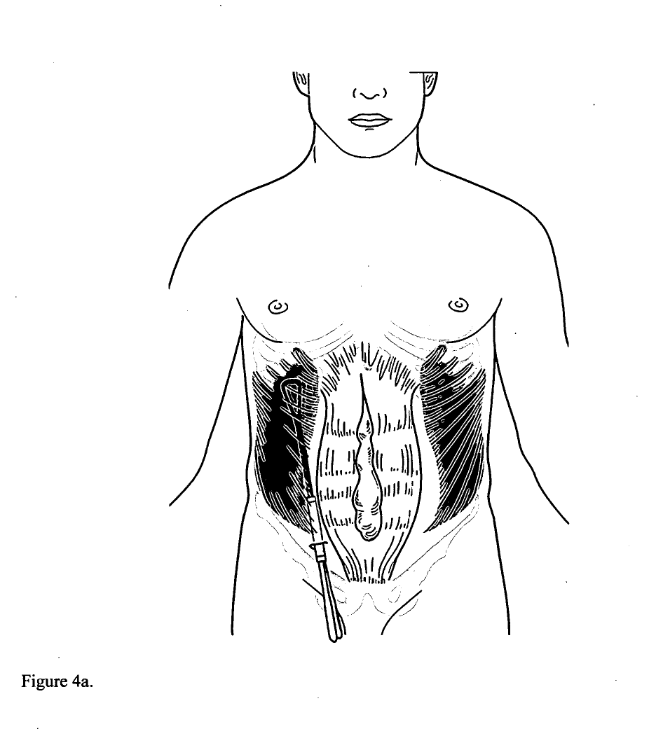

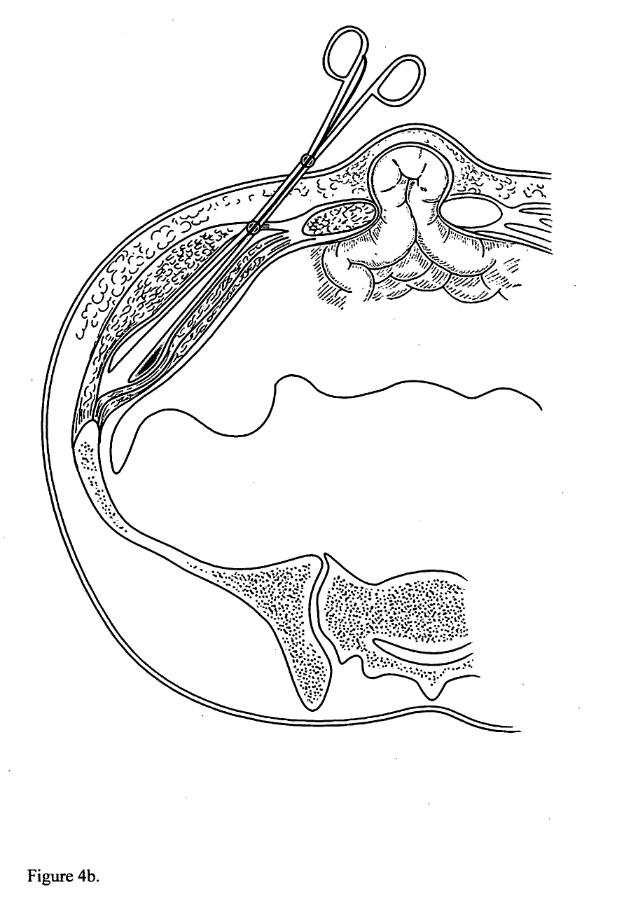

A final variation of this design provides for a mechanical coupling of the spoonbill plates to their spreader arms. In this way the initial dissection pocket is begun through a four to five centimeter incision in the inguinal region which allows insertion of the large spoonbill spreader plates into the dissected pocket between the EO and IO. Once the initial dissection is begun and an optical cavity is established, the large dissection plates are disconnected and left inside the cavity. The inguinal incision is closed over a 10 to 15 mm port and CO2 gas is insufflated. Second and third ports (10 mm) are inserted lateral to Spigal's line at points below the costal margin and above the anterior iliac spine. Now the instrument can be inserted through any of these ports and assembled to the dissection plates followed by completion of the dissection under endoscopic visualization (FIG. 4a and 4b).

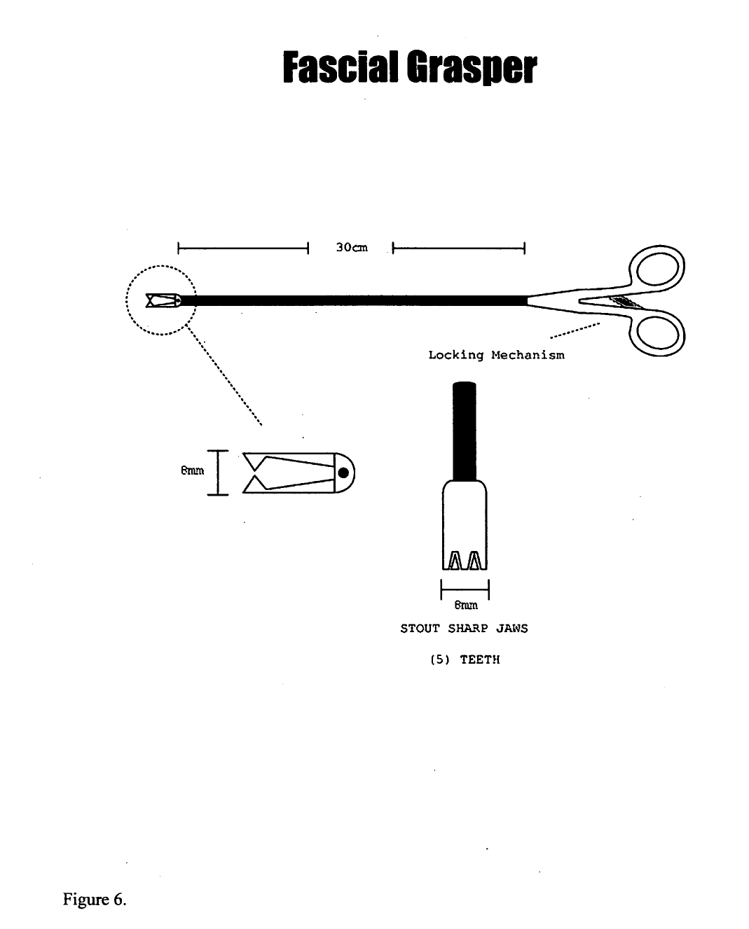

The fascial graspers 50 of the present invention are long endoscopic graspers. The graspers are modified to grasp and lock on to tissue as thick and tough as rectus fascia. They have five sturdy sharp teeth 52 that constitute a pair of stout jaws 54. It is approximately 8 mm. wide and can grab and lock on to an approximate thickness of 1 cm (FIG. 6). They are designed to insert into the peritoneal cavity through 10 mm ports and lock onto the hernia defect edge. Since this maneuver is performed laparoscopically, the edge of rectus fascia will be covered by the hernia sac and surrounding tissue that normally would be debrided away during an open repair. This makes the thickness of tissue to be grasped thicker and more slippery than what is encountered in open repairs. For this reason, the present grasper is much larger and stronger compared to conventional graspers to clamp on to and pull this edge of rectus fascia to the midline. Existing laparoscopic instruments have not been designed to grasp and hold the body wall; they are used to hold tissue and organs which are much more delicate.

The fascial stapler 60 coprising the present invention is designed for approximation of the mid line rectus fascia. With the stapler in the inguinal port and the fascial graspers holding the left and right rectus fascia together, the midline in the upper one third of the defect is stapled together. The stapler is then switched to the epigastric port and the lower two thirds of the midline is then stapled together. The device is long enough to reach the upper aspect of the defect from the inguinal port. The device requires a 15 mm. port and is designed to enter in a straight linear position. Once inserted to the peritoneal cavity, the instrument is adjusted to lock the distal staple cartridge into a 90 degree flexion. This allows the staples to approach the abdominal wall at a right angle. The handle is squeezed together thus firing the staple into the rectus fascia. The width and depth of the staple must be sufficient to grab and hold the thickness of the midline rectus fascia. Prior to full firing of the staple, a “cocked” position of the staple exists by partial squeezing of the stapler handles. This allows for partial exposure of the staple tips which can be used to hook the edge of one side of the mid line fascia and pull it over and engage the contra lateral side of fascia with the other partially exposed staple tip. After this is done, the stapler is fully fired. Existing endoscopic staplers and tackers have been designed to function in a linear fashion for such procedures as laparoscopic inguinal hernia repairs. This new procedure will require a new generation of stapling devices aimed at the midline abdominal wall. In order to perform a secure closure of the linea alba, staples must enter the left and right midline fascia at right angles to the abdominal wall. Since the instrument enters the abdominal wall and must turn on itself to approach the midline at right angles, there is a provision for flexion of the staple cartridge distally. This use of a novel endoscopic stapler will also call for a larger staple design. Since the midline abdominal wall is thick and bulky, the depth of the staple must be sufficient to engage and hold both sides. In addition it must penetrate the posterior rectus sheath and have enough length to also engage the anterior rectus sheath prior to closure of the staple.

The present invention includes several apparatus as previously described as well as subsequently indicated; a mechanical dissector, a grasper, and a stapler each comprised of a plurality of components.

FIG. 1 represents the case of an existing incisional hernia. In the preferred embodiment the midline is reconstructed thus restoring form and function to the abdominal wall and a Biosurgical Prosthetic Rectus Sheath is placed intra peritoneal and anchored to the lateral abdominal wall to re enforce the repair and balance abdominal wall tensions. But first a fascial release must be performed in order to bring the rectus muscle and linea alba together without undue tension. This is accomplished through a Components Separation Technique using a minimally invasive approach (MICST). This release is required due to the lateral retraction vectors which come into play in the development of incisional hernias.

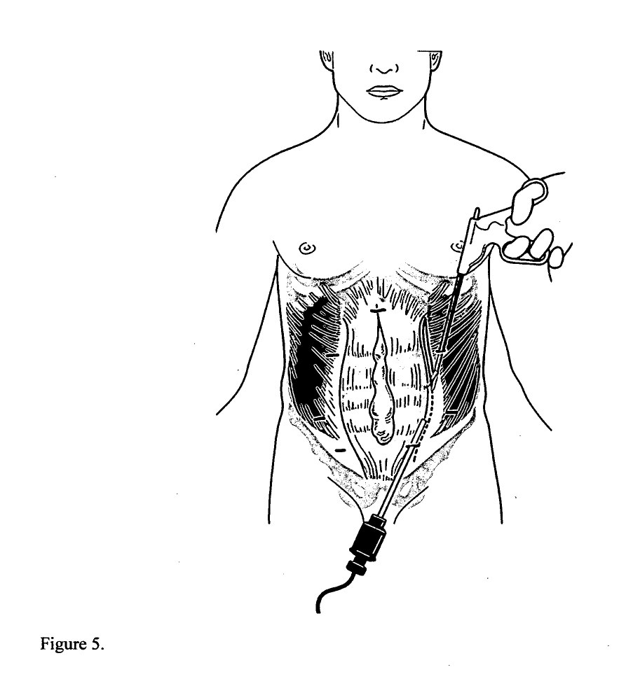

MICST is begun with a small horizontal incision just above the external inguinal ring. This anatomic region is completely familiar to all general surgeons (the same incision is used for open inguinal hernia repairs) and consistent in all body types despite the size of the ventral hernia. A small vertical incision through the deep fascia is then made just above and lateral to this point. This opens the fascial cleft between the External Oblique (EO) and Internal Oblique (IO). The surgeon's index finger performs the initial blunt dissection in this plane and verifies correct position by the ease of separation of the plane laterally but the inability to bluntly dissect medially past Spigal's line (the line of fusion of the EO and IO fascia at the lateral border of the rectus muscle—the so called semi-lunar line). Next a MICST mechanical blunt dissector (FIG. 3) is inserted into this fascial plane and an initial optical cavity is developed. The spoonbill dissector plates are detached and a 10 to 15 mm laparoscopic port is inserted into the cavity and the space insulflated with CO2 gas. Two additional ports (10 mm) are inserted under endoscopic visualization four centimeters lateral to the semi-lunar line, below the costal margin and above the iliac crest. The mechanical blunt dissector is now re inserted into the cavity and the dissection plates are reassembled under endoscopic visualization. Blunt dissection is continued until the limits of the dissection are reached (Spigal's line medially, above the costal margin and below the infra-mammary fold superiorly, lumbar region laterally and inguinal region inferiorly)—FIGS. 4a and 4b. This step is then repeated on the contra-lateral side of the abdomen. Next, the mechanical dissectors are removed. A 10 mm. laparoscope is then inserted into the inguinal port. Endoscopic electro-cautery scissors are inserted through the 10 mm ports and used to divide the EO fascia two centimeters lateral to the semi-lunar line. The superior release of this fascia is performed by inserting the endoscopic scissors through the inferior “four corners” incision, while the inferior fascial release is performed through the superior “four corners” trocar (FIG. 5). These steps are then repeated on the contra-lateral side. This is a safe, a-vascular region so this maneuver is quick and easy to perform. These maneuvers constitute a Minimally Invasive Components Separation.

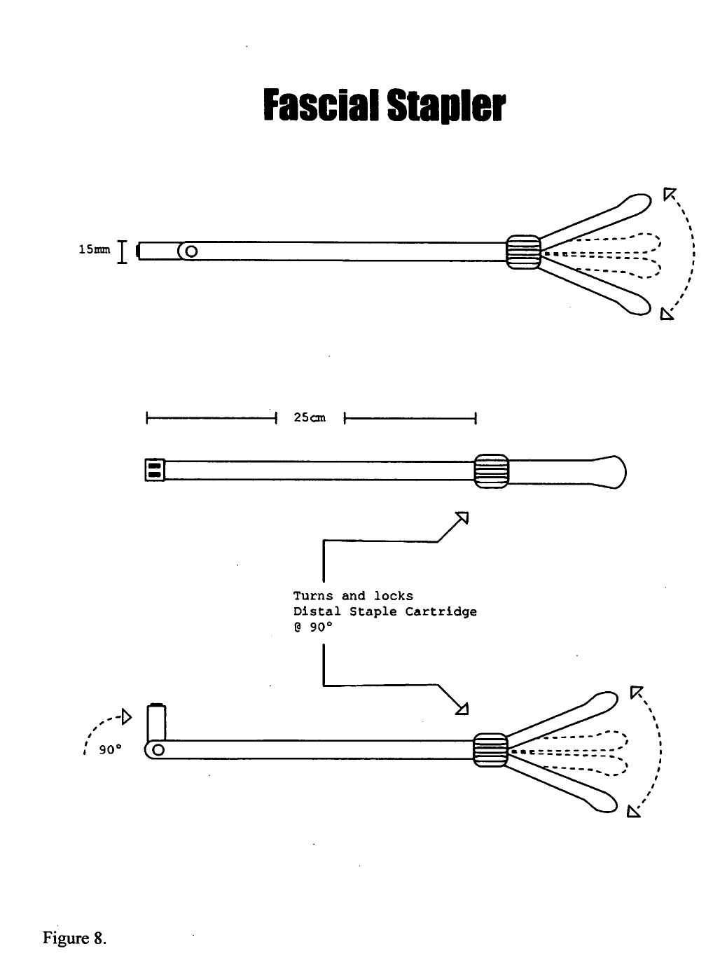

Part B of the minimally invasive ventral hernia repair (MIVHR) involves the laparoscopic closure of the midline using new laparoscopic fascial graspers (FIG. 6) and a new laparoscopic rectus fascial stapler (FIG. 8). Finally, the Biosurgical Rectus Sheath is stapled in place along the posterior side of the abdominal wall, and its lateral and superior edges are tied to the abdominal wall at the laparoscopic access ports.

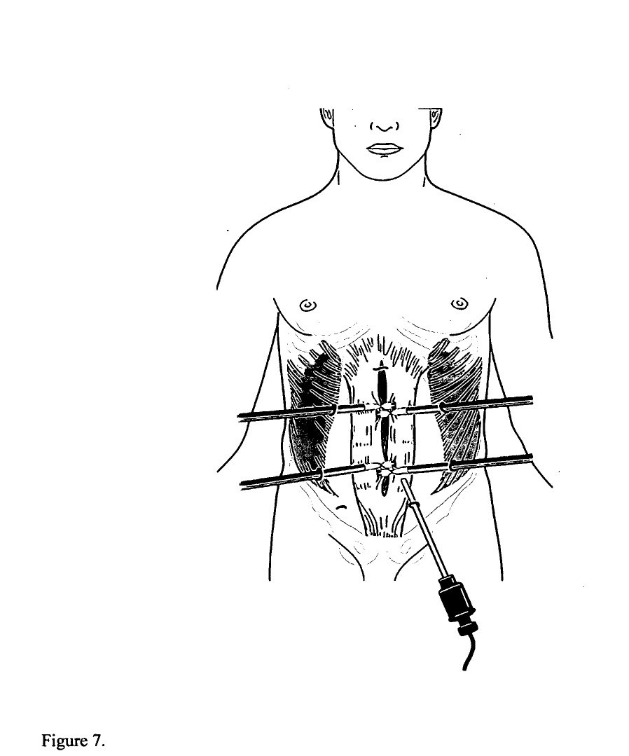

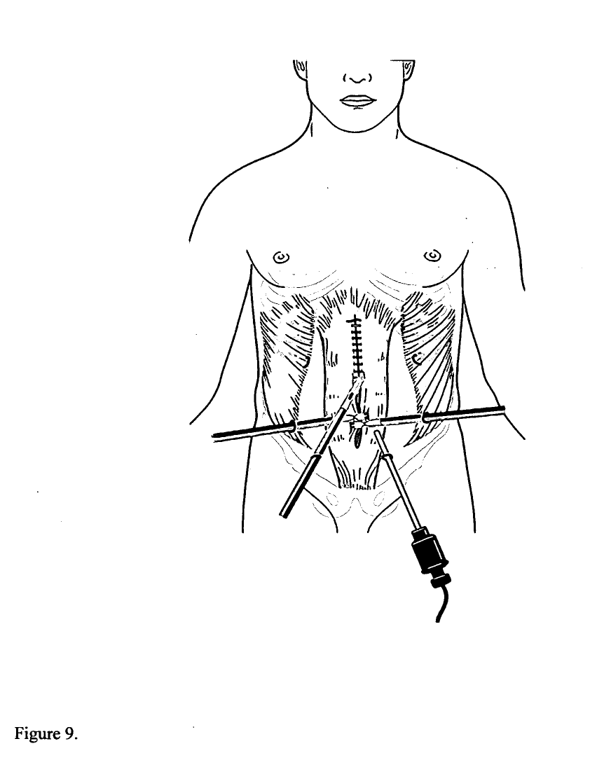

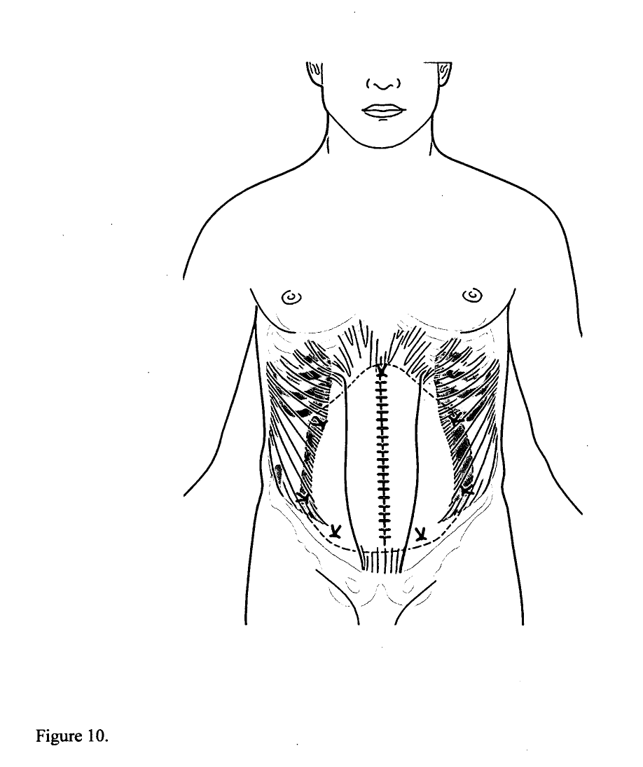

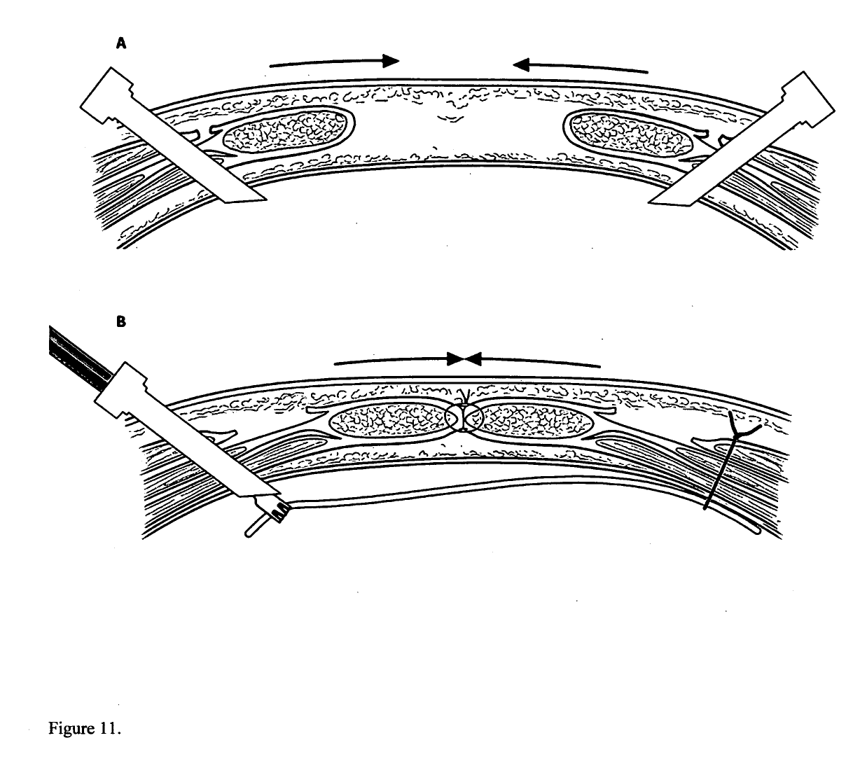

This is accomplished by inserting the 15 mm. trocars through the remaining layers of the abdominal wall in a more medial and superior direction. The peritoneal cavity is then insufflated with CO2 gas. A third 15 mm. trocar is placed in the epigastric midline to establish a triangulation of large ports. The 10 mm. trocar ports, at the four corners of the anterior abdominal wall, are then inserted through the remaining layers into the peritoneal cavity. A 10 mm laparoscope is inserted through one of the inguinal ports and a Rectus Fascial Stapler through the other. Fascial graspers are inserted into all four corners and placed across the midline in order to pull the contra-lateral midline to the center (FIG. 7). The Rectus Fascial Stapler is then used to staple the midline fascia together in the upper half of the defect (FIG. 9). The Rectus Fascial Stapler is then switched to the epigastric port and the lower half of the defect is stapled together. Next, the Biosurgical Rectus Sheath is introduced into the peritoneal cavity and unfurled with the anti adhesion side towards the viscera. It is centered over the midline and stapled to the posterior rectus sheath. The lateral wings of the mesh are then anchored to the full thickness of the lateral abdominal wall through the “four corners” trocar sites, after they are pulled back and re inserted through the advanced abdominal layers (FIG. 10). These sutures are tied through the lateral edge of the released External Oblique fascia for maximal strength (FIG. 11). In this way the tension of the intra abdominal cavity is transmitted to the lateral components and offloads tension from the midline repair. Closure of the fascia at the large ports also incorporates the mesh to insure anchoring of the mesh to the abdominal wall superiorly and inferiorly. The lateral anchoring of the mesh is designed to be closed under tension so that it will be taught when insulflation is released. Reinforcement of the midline is provided through the mesh inlay's baffling nature, and accomplishes the ultimate in achieving the principle of “overlapping” the defect. It also protects against the potential weakness in the region of the components separation relaxing incision. This is done by covering this area with the lateral wings of the mesh (from the intra abdominal side) and anchoring it to all four layers of the abdominal wall. Finally, three anchoring sutures are placed along the old midline scar as a mass closure of the linea alba (including the mesh layer).

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed as being new and desired to be protected by Letters Patent of the United States is as follows:

1. A new and improved set of instrument instruments and method of their use in laparoscopic minimally invasive ventral hernia repair comprising, in combination:
a mechanical blunt dissector capable of performing a blunt dissection of a space between an External Oblique (EO) muscle and an Internal Oblique (IO) muscle wherein the dissector includes a proximal end 20 and distal end 22 operable associates at a hinge point 24, the distal end of the dissector includes a blunt spoonbill shaped set of plates, the plates haing an open center providing for improved endoscopic visualization during a dissection, the proximal end of the dissector is connected to the distal end by elongate arms, the proximal end includes a pair of hand grips, the hinge point is formed in the elongate arms such that in use the handles are kept outside a body allowing the spoonbill dissector plates to be positioned within a body for blunt dissection inside the body, between the distal end and hinge point is provide a joining region of the arms to the grips has a narrow waist to allow wide spreading of the arms through a minimal incision, the arms being arched to give strength to the device;
fascial graspers comprising long endoscopic graspers modified to grasp and lock on to tissue as thick and tough as rectus fascia, the graspers including sturdy sharp teeth associated with stout jaws for grabbing and locking on to an approximate thickness of 1cm, the graspers are designed to insert into the peritoneal cavity through ports and lock onto the hernia defect edge;
a fascial stapler comprising adapted for approximation of the mid line rectus fascia such that the stapler is long enough to reach the upper aspect of the defect from a port and adjusted to lock a distal staple cartridge into a 90 degree flexion for allowing staples to approach the abdominal wall at a right angle.

2. The new and improved new and improved set of instrument instruments and method of their use in laparoscopic minimally invasive ventral hernia repair as set forth in claim 1 wherein blunt dissector includes a tapered blunt distal tip to the spoonbill element not unlike like a duck bill shape for advancing the mechanical dissector forward into the loose areolar connective tissue that exists between the EO and IO muscles;

3. The new and improved new and improved set of instrument instruments and method of their use in laparoscopic minimally invasive ventral hernia repair as set forth in claim 2 wherein the blunt dissector includes separating means for the hinge of the hand grip arms from the hinge of the spoonbill dissector plate arms with an elongated tubular structure for allowing a smaller incision site in the inguinal region and better maneuverability of the instrument through this incision.

4. The new and improved new and improved set of instrument instruments and method of their use in laparoscopic minimally invasive ventral hernia repair as set forth in claim 3 and further including a mechanical coupling means of the spoonbill plates to their spreader arms.

5. A new and improved set of instrument instruments and method of their use in laparoscopic minimally invasive ventral hernia repair comprising the following steps, in combination:
Providing a fascial release must be performed in order to bring the rectus muscle and linea alba together without undue tension through a Components Separation Technique using a minimally invasive approach (MICST);
Beginning MICST with a small horizontal incision just above the external inguinal ring;
Providing a small vertical incision through the deep fascia is then made just above and lateral to this point thereby opening the fascial cleft between the External Oblique (EO) and Internal Oblique (IO);
Next, the surgeon's index finger performs the initial blunt dissection in this plane and verifies correct position by the ease of separation of the plane laterally but the inability to bluntly dissect medially past Spigal's line (the line of fusion of the EO and IO fascia at the lateral border of the rectus muscle—the so called semi-lunar line);
Next a MICST mechanical blunt dissector is inserted into this fascial plane and an initial optical cavity is developed;
Next spoonbill dissector plates are detached and a 10 to 15 mm laparoscopic port is inserted into the cavity and the space insulflated with CO2 gas;
Next two additional ports (10 mm) are inserted under endoscopic visualization four centimeters lateral to the semi-lunar line, below the costal margin and above the iliac crest;
Next, the mechanical blunt dissector is re-inserted into the cavity and the dissection plates are reassembled under endoscopic visualization;
Next, blunt dissection is continued until the limits of the dissection are reached;
Next, the previous step is then repeated on the contra-lateral side of the abdomen;
Next, the mechanical dissectors are removed;
Next, a 10 mm. laparoscope is then inserted into the inguinal port and Endoscopic electro-cautery scissors are inserted through the 10 mm ports and used to divide the EO fascia two centimeters lateral to the semi-lunar line;
Next, the superior release of this fascia is performed by inserting the endoscopic scissors through the inferior “four corners” incision, while the inferior fascial release is performed through the superior “four corners” trocar;
Next, these steps are then repeated on the contra-lateral side;
Next, involves the laparoscopic closure of the midline using new laparoscopic fascial graspers and a new laparoscopic rectus fascial stapler and the Biosurgical Rectus Sheath is stapled in place along the posterior side of the abdominal wall, and its lateral and superior edges are tied to the abdominal wall at the laparoscopic access ports this is accomplished by inserting the 15 mm. trocars through the remaining layers of the abdominal wall in a more medial and superior direction;
Next, the peritoneal cavity is then insufflated with CO2 gas and a third 15 mm. trocar is placed in the epigastric midline to establish a triangulation of large ports;
Next, the 10 mm. trocar ports, at the four corners of the anterior abdominal wall, are then inserted through the remaining layers into the peritoneal cavity and a 10 mm laparoscope is inserted through one of the inguinal ports and a Rectus Fascial Stapler through the other;
Next, fascial graspers are inserted into all four corners and placed across the midline in order to pull the contra-lateral midline to the center;
Next, the Rectus Fascial Stapler is then used to staple the midline fascia together in the upper half of the defect and the Rectus Fascial Stapler is then switched to the epigastric port and the lower half of the defect is stapled together;
Next, the Biosurgical Rectus Sheath is introduced into the peritoneal cavity and unfurled with the anti adhesion side towards the viscera and it is centered over the midline and stapled to the posterior rectus sheath and the lateral wings of the mesh are then anchored to the full thickness of the lateral abdominal wall through the “four corners” trocar sites, after they are pulled back and re inserted through the advanced abdominal layers such that these sutures are tied through the lateral edge of the released External Oblique fascia for maximal strength so the tension of the intra abdominal cavity is transmitted to the lateral components and offloads tension from the midline repair whereby closure of the fascia at the large ports also incorporates the mesh to insure anchoring of the mesh to the abdominal wall superiorly and inferiorly and the lateral anchoring of the mesh is designed to be closed under tension so that it will be taught when insulflation is released whereby reinforcement of the midline is provided through the mesh inlay's baffling nature, and accomplishes the ultimate in achieving the principle of “overlapping” the defect such that it also protects against the potential weakness in the region of the components separation relaxing incision in as much as this is done by covering this area with the lateral wings of the mesh (from the intra abdominal side) and anchoring it to all four layers of the abdominal wall, and finally, three anchoring sutures are placed along the old midline scar as a mass closure of the linea alba (including the mesh layer);
Whereby, the midline is reconstructed thus restoring form and function to the abdominal wall and a Biosurgical Prosthetic Rectus Sheath is placed intra peritoneal and anchored to the lateral abdominal wall to re enforce the repair and balance abdominal wall tensions.
